(19) United States
(12) Patent Application Publication
(10) Pub. No.: US 2015/0327910 A1
(43) Pub. Date: Nov. 19, 2015

(54) Surgical Forceps with Spring Member Having an Adjustable Position

(71) Applicant: Gerard Michael Brooke, Tetbury (GB)

(72) Inventor: Gerard Michael Brooke, Tetbury (GB)

(21) Appl. No.: 14/653,595

(22) PCT Filed: Dec. 18, 2013
(86) PCT No.: PCT/GB2013/053335

§ 371 Date: Jun. 18, 2015

Related U.S. Application Data
(60) Provisional application No. 61/740,664, filed on Dec. 21, 2012.

(30) Foreign Application Priority Data
Dec. 20, 2012 (GB) 1223090.0

(51) Int. Cl. A61B 18/14; A61B 17/28; A61B 17/30

(52) U.S. Cl. CPC A61B 18/1442; A61B 17/30; A61B 17/28; A61B 17/2841; A61B 2017/2845; A61B 2018/00095

(57) ABSTRACT

Surgical forceps, for example electrosurgical forceps, are disclosed which comprise a pair of arms, each extending from a connecting hinge or member at a rearward end of the forceps to a tip at the forward end of the forceps. A spring or pivot member is disposed between the forward and rearward ends of the forceps and engages the arms. The axial position of the spring or pivot member is adjustable.

### FIELD OF THE INVENTION

This invention relates to surgical forceps and in particular, but not exclusively, to forceps for electrosurgery.

### BACKGROUND OF THE INVENTION

Surgical forceps are generally handheld hinged instruments which are used to grasp or hold objects such as biological tissue.

Electrosurgery comprises a method of surgery in which a high frequency electric current is applied to biological tissue in order to cut, coagulate, desiccate and/or fulgurate the tissue. In particular, electrosurgical devices are commonly used during surgery in order to stop bleeding by using an alternating current to directly heat tissue and thereby reduce blood loss and/or improve surgical vision.

Two primary types of electrosurgical device are known, namely bipolar and monopolar devices.

In monopolar arrangements the electrosurgical device is provided with an active electrode and a return electrode is attached to the patient. The electric current flows from the active electrode into the body and returns through the return electrode (which is connected to an earthing circuit). The current density decreases rapidly with distance away from the electrode such that the heating of tissue is localised to the tip of the electrosurgical device.

In bipolar devices, a pair of electrodes, for example the tips of forceps, are each connected to the supply circuit and no return electrode is required. When tissue is engaged by or proximal to the pair of electrodes, the high frequency electric current flows through the device and tissue providing a localised heating of the tissue.

Known forceps for use in electrosurgery are typically provided in a range of sizes such that a surgeon may select their preferred forceps for a particular application. In particular, a surgeon will generally choose differing lengths of forceps depending upon the tissue which is to be accessed and the degree of control that is required. It would be advantageous to provide a single pair of forceps which could be suitable for a variety of tasks such that the surgeon can easily switch between different tasks and/or such that the need to prepare numerous different instruments for a single surgical operation is reduced.

### SUMMARY OF INVENTION

According to a first aspect of the present invention, there is provided surgical forceps comprising a pair of arms, each extending from a connecting hinge at a rearward end of the forceps to a tip at the forward end of the forceps and a spring member disposed between the forward and rearward ends of the forceps and engaging the arms and wherein the axial position of the spring member is adjustable. Advantageously, the axial movement of the spring member enables the closing force and the feel of the forceps to be adjusted. The forceps may be for electrosurgery.

The spring member may provide a pivot. For example, the spring member may be the main pivot for the arms. The hinge may be a connecting member between the arms. The hinge may primarily act as a flexible connecting means between the arms (but may, for example, be of minimal function as a pivot point during use). For example, the spring member may be the main load bearing pivot or hinge point of the forceps. It will be appreciated that the flexibility of the spring member may be selected in accordance with the desired mechanical action of the forceps.

The tips of the arms are typically arranged for engaging tissue during surgery.

The spring may be generally transverse to the longitudinal axis of the forceps. For example, the spring is generally arranged across the gap between the arms of the forceps.

The connecting hinge at the rearward end of the forceps may be arranged to align the arms of the forceps and ensure that the opposed tips of the arms remain aligned.

The forceps may be bipolar forceps.

The spring member may, for example, comprise a resilient member (for example a U-shaped resilient member). The limbs of the U-shaped member may each be arranged to engage one of the arms of the forceps. The spring member may be formed from an insulating material.

The spring member may further comprise two body portions. Each body portion may be arranged to engage one of the arms. For example, the body portions may each be adapted to slidably engage one of the arms.

The spring member may further comprise a pair of opposing finger receiving portions each arranged on the outer surface of one of the arms of the arms. For example the finger receiving portions may each be provided on one of the body portions. Accordingly, the finger-receiving portions may generally be arranged to be gripped between the finger and thumb of a surgeon when using the forceps. The finger-receiving portions may be inwardly compressed to close the tips of the forceps.

The spring member may further comprise a gripping surface. For example a gripping surface may be provide on the sides of the spring member, for example on the sides of the body portions. member. The gripping surfaces are adapted to be used for adjusting the axial position of the spring member.

The spring member may further comprise a switch which may, for example, be arranged to activate the high frequency current. For example, the switch may be provided on one of the body portions, for example on the finder recebing portion of one of the body portions. The forceps may, for example, further include a PCB assembly for the switch. The switch may be a membrane switch.

The spring member and arms may be provided with complimentary engagement formations. The complimentary engagement formations may, for example, comprise a slot in each arm and a pair of carriage members on the spring member. Each carriage member may be sized to be received into the slot on one of the arms. The complimentary engagement formations may, for example, be arranged to define the range of axial adjustment of the spring member. The slot in each arm may be provided on the inner surface.

The complimentary engagement formations may be arranged to provide indexed positions for the spring member. For example, a rack may be provided on at least one of the arms (for example, within the slot) and a complimentary profile tooth may be provided on the spring member (for example, on the carriage member). The tooth may be provided on a resilient portion of the spring member (for example, a resilient portion of the carriage member). Thus, the tooth may be arranged to deflect over the projections of the rack and engage the rack recesses. Accordingly, the spring member may be arranged to click across a range of index positions. The rack may be on the inner surface of the arm. The tooth may be outwardly projecting.

The connecting hinge may be provided with an electrical supply, and may, for example, be connected to a PCB.

The forceps may be provided with thermally conductive tips, which may be formed from aluminium. This is considered novel and inventive in its own right and, therefore, according to a second aspect, the present invention provides forceps for electrosurgery comprising a pair of arms comprising elongate members each extending from a connecting hinge at a rearward end tip at a forward end; and wherein the arms comprise thermally conductive tips attached to the forward end. The tips are discrete and are attached to the arms by any suitable means.

The tips may be formed from a material having high thermal conductivity. For example, the tips may be metallic. The material may, for example, have a thermal conductivity of at least 15 watts per meter kelvin (Wm⁻¹K⁻¹). For example, the material may have a thermal conductivity of at least 100 watts per meter kelvin (wm⁻¹k⁻¹). For example, the tips may be formed from aluminium or an aluminium alloy.

Advantageously, the use of separately formed tips enables the strength to be carefully controlled overcoming the disadvantages of the flexibility of aluminium while providing improved heat transfer and by compatibility in comparison to conventional (for example, stainless steel) forceps.

The metal tips may be provided with a non-stick coating. For example, the tips may be coated with PTFE or Diamond-Like Carbon (DLC). The non-stick coating may have a high thermal conductivity. The electrical conductivity of the coating (or even the tips) is of lesser importance due to the high frequency current used in electrosurgery.

The non-stick coating may be a bio-compatible coating.

The arms of the forceps may comprise a plastic outer body and the tips may be embedded into the forward end of the body. For example, the plastic body may be over-moulded onto the tips. The arms may be provided with an embedded conductor for supply electrical current to the tips. The embedded conductor may also be a structural component of the arms, for example a stiffener. Alternatively, a separate stiffening member may be provided. For example, the arms may comprise a metal body which is over-moulded with an insulating plastic material. The tips may be received within and/or attached to the metal body.

The arms may comprise a stiffener and a rearward section of the tips interconnects with the stiffener. For example the rearward section of the tips may be received within the stiffener. Alternatively, or additionally, the rearward tips may be attached to the stiffener.

Whilst the invention has been described above, it extends to any inventive combination of features set out above or in the following description or drawings.

### BRIEF DESCRIPTION OF THE DRAWINGS

A specific embodiment of the invention will now be described in detail, by way of example only, and with reference with the accompanying drawings in which

FIG. 1 is a schematic three-dimensional view of forceps for electrosurgery in accordance with an embodiment of the invention;

FIG. 2 is a schematic three-way projection of the embodiment shown in FIG. 1;

FIG. 3 is a schematic cross-section through A-A of FIG. 2A; and

FIG. 4 is a schematic three-dimensional partial cut away of the tip arrangement of FIG. 1.

### DETAILED DESCRIPTION OF EMBODIMENT

Front as used herein will be understood to refer to the end of the forceps (or components thereof) which, in use, are closest to the tissue on which a procedure is being carried out (i.e. the end which is facing the patient). Rear as used herein will be understood to refer to the end of the forceps (or components thereof) which, in use, are furthest from the tissue (i.e. the end which is facing the surgeon). Forward and rearward will, likewise, be understood to refer to the directions orientated towards the front and rear of the forceps.

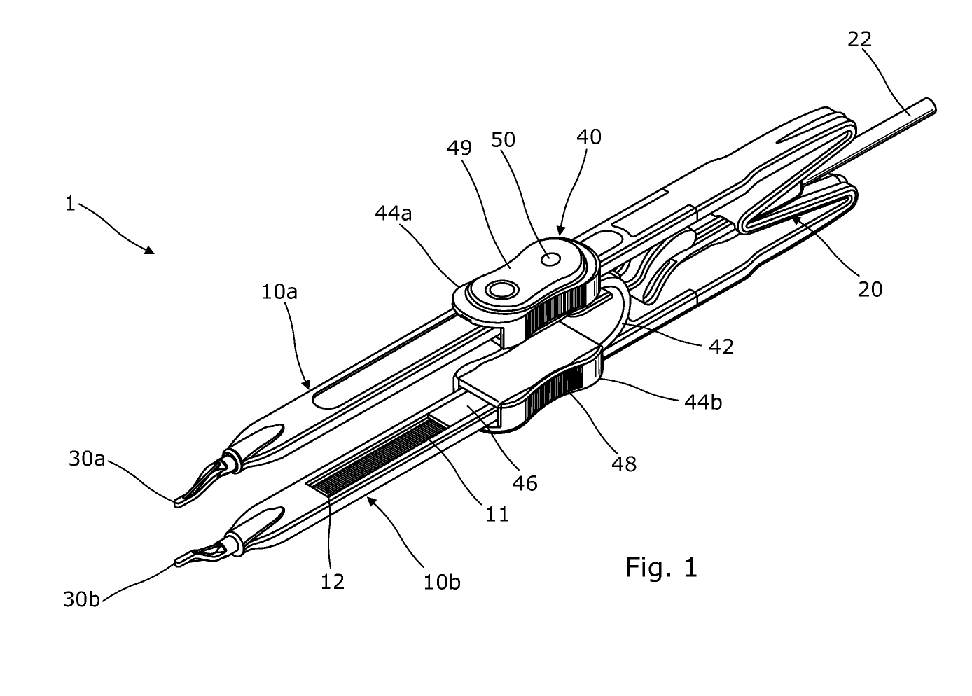

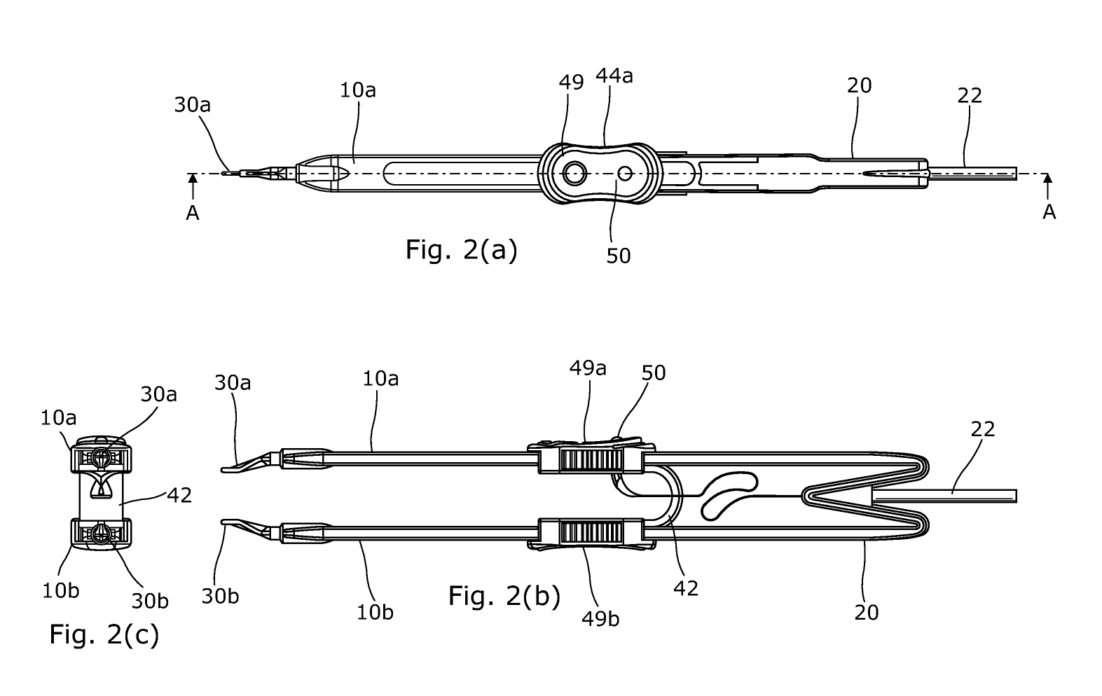

Forceps 1 for electrosurgery in accordance with an embodiment of the invention are shown in FIGS. 1 to 3. The forceps comprise a pair of elongate arms 10A and 10B which extend from a hinge 20 at a rearward end to tips 30A and 30B at a forward end.

A spring member 40 is provided between the hinge 20 and tips 30, and extends generally transversely across the space between the arms 10A and 10B. The spring member 40 will be described in further detail below.

The arms 10 are provided with an outer insulating plastic coating and have a stiffener 13 running along their length which may, for example, be formed from steel. Hinge 20 connects the arms 10A and 10B and ensures that the tips 30A and 30B are aligned such that they may be precisely closed, in use, across tissue. The hinge 20 may biases the forceps to the open position (as shown in the figures) such that, in use, they may be deflected into a closed position. Alternatively, this bias may be provided by the spring member 40.

As will be explained below, the main pivot point (and therefore “hinge”) between the arms may be provided by the spring member 40 and the rearward hinge 20 may only be a connecting means between the arms 10A and 10B. The primary function of the hinge 20 may therefore as a supporting means for the cable entry. The hinge 20 may additionally or alternatively be provided in order to provide ergonomic balance during use of the forceps.

The hinge 20 is provided with a supply 22 for receiving high frequency (for example 500 MHz) alternating electrical current. A connector wire 24 is embedded within hinge 20 for supplying the current from the supply to the arms of the forceps.

The tips 30A and 30B of the arms 10A and 10B are formed from discrete sections of pressed aluminium (although a skilled person will appreciate that the aluminium may be formed by any convenient means, for example, milling, etc.). The tips are provided with a non-stick coating which may, for example, be PTFE or Diamond-Like Carbon (DLC). Aluminium tips are desirable due to the good thermal conductivity. The thermal conductivity of the tips may enable the tips to act, in part, as a heat sink so as to assist with heat transfer away from tissue during use. Advantageously, the use of discrete tip sections enables the strength of the aluminium to be precisely controlled and reduces any unwanted effects of the flexibility of aluminium.

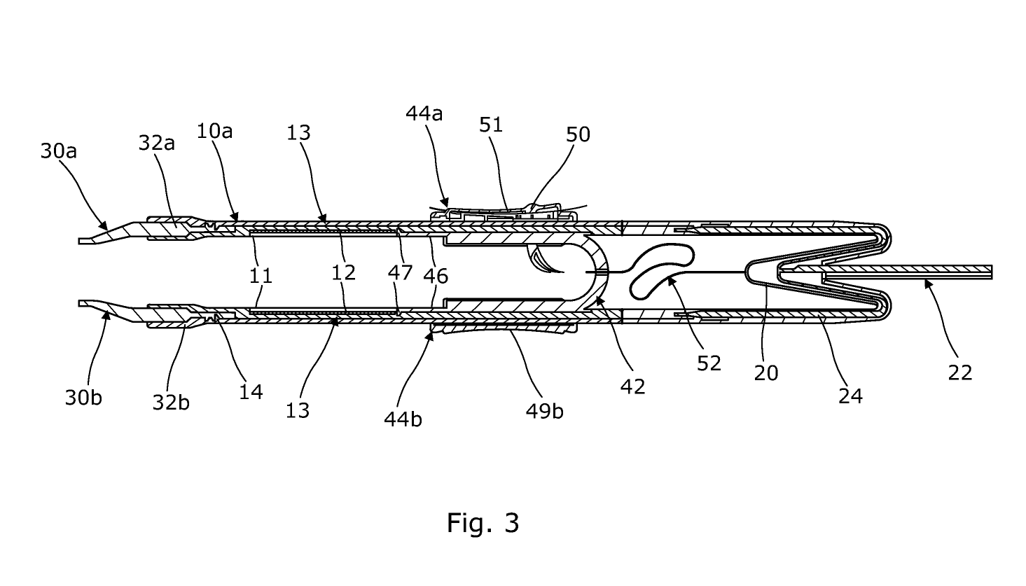

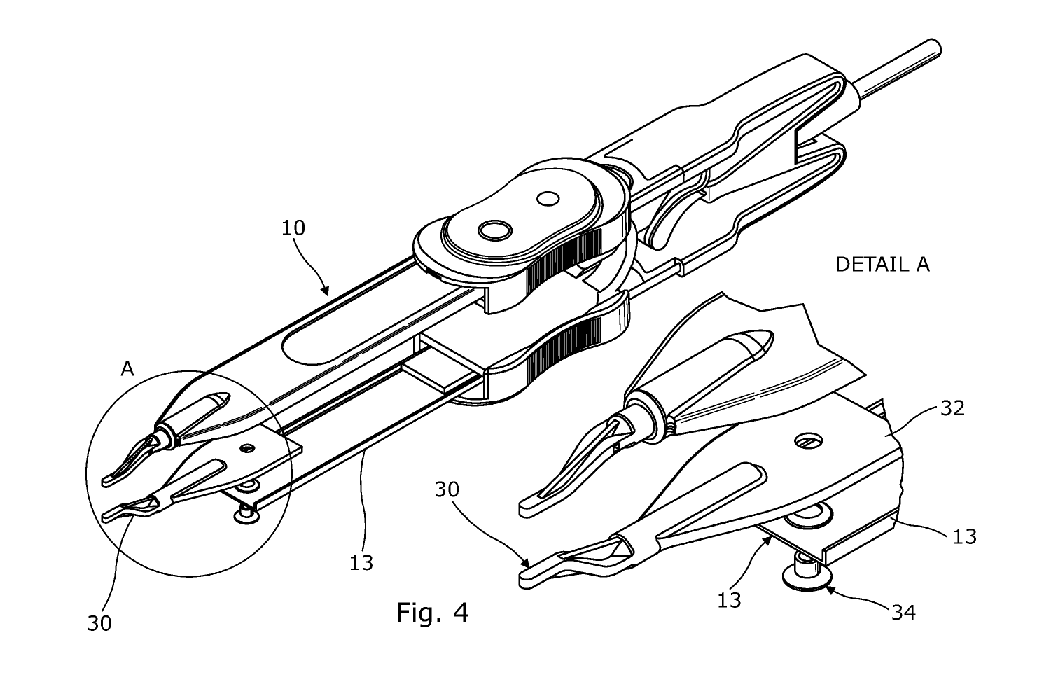

As seen in FIG. 3, the rear portion 32 of the tips 30 extends rearwardly into the body of the arms 10 such that the tips are partially embedded within the arms. The plastic outer surface of arm 10 is over-moulded onto the rear section 32. The rear section is provided with a shaped profile which interconnects with a complimentary shaped profile 14 of the stiffener 13 within the body of the arm 10. For example, as best seen in FIG. 4, the stiffener 13 may have a U-shaped cross section such that it defines a channel into which the rear portion 32 of the tip 30 is received. The tip may be secured by any suitable means, for example a rivet 34. The stiffener 13 and tip 32 are subsequently over moulded with an electrically insulating layer of plastic. This advantageously provides a secure engagement of the discrete tip 10. It will be appreciated that the stiffener 13 may typically be formed from a metallic material and may conveniently also function as an electrical conductive path to the tips 30.

The spring member 40 generally comprises a resilient member in the form of a U-shaped compression spring 42 which is positioned between the arms 10A and 10B. The limbs of the U-shaped member are integral with two body portions 44A and 44B of the spring member 40 each of which is arranged to be carried on one of the respective arms 10A and 10B of the forceps. The body portions include finger-receiving portions 49A and 49B arranged on the outer surface of the arms 10A and 10B and intended to be gripped between the forefinger and thumb of a surgeon in use (such that the surgeon may squeeze the arms 10A and 10B to close the opposing tips 30A and 30B). The body 44A and 44B is generally arranged to surround the local portion of its respective arm 10A, 10B such that it slidably engages the arm and enables the spring 42 to be axially moved relative to the arms 10A and 10B. The inner portion of each arm (i.e. the portion facing the opposing arm) is provided with a recessed slot 11, the surface of which is provided with a rack 12. The spring member 40 is provided with a carriage 46 which is sized and shaped so as to be received into the slot 11 of the arm 10. Thus, the carriage 46 and slot 11 provide complimentary engagement formations which define the axial range of movement of the spring member 40. Each carriage 46 is provided with an outwardly projecting tooth 47.

One of the body portions 44A is further provided with a switch 50. The switch is arranged to engage a membrane switch which is located within the finger-receiving portion 49 of the body 44A as part of a PCB sub assembly 51. As such, the body 44A has a hollow two-part construction. The PCB sub-assembly 51 is connected to a flexible PCB 52 which is in turn connected to the supply 22 within the hinge 20.

In use, a surgeon may select the axial position of the spring member 40 using gripping surfaces 48 which are provided on the side edges of the bodies 44A and 44B. The spring member 40 is slid to a desired axial position and the tooth 47 of the carriage 46 is able to resiliently deform so as to pass over the projections of the rack 12 so as to provide a series of indexed positions for the spring member 40. Typically, an audible click will be heard as the tooth 47 of the carriage 46 translates across the rack thereby providing the user with a degree of feedback. The user selects the desired position based upon the location of the finger-receiving portions 49A and 49B so that the arms 10 extend beyond the finger-receiving portion 49 to the tip 30 by a chosen extent. The spring 42 acts as the main pivot point of the forceps (and additionally may 42 provides a constant spring force at the selected position) and is moved in conjunction with the finger-receiving portions such that the lever arm of the forceps are adjusted; thus, the feel and feedback of the forceps is controlled. It will be appreciated that in acting as the main pivot point the spring 42 is effectively acting as a hinge between the arms 10A and 10B. The hinge 20 is primarily acting as a flexible connection between the arms 10A and 10B but does not need to provide any substantial load bearing during use.

While the invention has been described above with reference to a preferred embodiment, it will be appreciated that various changes or modifications may be made without departing from the scope of the invention as defined in the appended claims.

For example, in some embodiments the spring member may even be substantially rigid. For example, opening and closing of the forceps may be achieved via the flexibility of the arms of the forceps rather than of the spring member. Thus, in embodiments the spring member may be a pivot member and the spring may be a pivot. Therefore, embodiments of the present invention may provide surgical forceps comprising a pair of arms, each extending from a connecting member at a rearward end of the forceps to a tip at the forward end of the forceps and a pivot member disposed between the forward and rearward ends of the forceps and engaging the arms and wherein the axial position of the pivot member is adjustable. Said embodiment may be combined with any features described herein.

What is claimed is:

1. Surgical forceps comprising:
a pair of arms, each extending from a connecting hinge at a rearward end of the forceps to a tip at the forward end of the forceps; and
a spring member disposed between the forward and rearward ends of the forceps and engaging the arms and wherein the axial position of the spring member is adjustable.

2. Forceps as claimed in claim 1, wherein the forceps are electrosurgical forceps.

3. Forceps as claimed in claim 1, wherein the spring member is the main pivot for the arms in use.

4. Forceps as claimed in claim 1, wherein the spring member comprises a resilient U-shaped member, the limbs of the U-shaped member each engaging one of the arms.

5. Forceps as claimed in claim 1, wherein the spring member further comprises two body portions each adapted to slidably engage one of the arms.

6. Forceps as claimed in claim 4, wherein the body portions each comprise a pair of opposing finger receiving portions each arranged on the outer surface of one of the arms of the arms.

7. Forceps as claimed in claim 5, wherein at least one of the body portions further comprises includes a switch.

8. Forceps as claimed in claim 1, wherein the spring member further comprises a gripping surface.

9. Forceps as claimed in claim 1, wherein the spring member and arms are provided with complimentary engagement formations.

10. Forceps as claimed in claim 9, wherein the complimentary engagement formations comprise a slot in each arm and a pair of carriage members on the spring member, each carriage member being sized to be received into the slot on one of the arms.

11. Forceps as claimed in claim 9, wherein the complimentary engagement formations provide indexed positions for the spring member.

12. Forceps as claimed in claim 11, wherein the indexing positions are provided by a rack on at least one of the arms and a complimentary profiled tooth on the spring member.

13. Forceps as claimed in claim 1, wherein the fixed hinge further comprises an electrical supply.

14. Forceps as claimed in claim 1, wherein the arms further comprise thermally conductive tips.

15. Forceps for electrosurgery comprising:
a pair of arms comprising elongate members each extending from a connecting hinge at a rearward end of the forceps to a tip at a forward end; and wherein
the arms further comprise thermally conductive tips attached at the forward end.

16. Forceps as claimed in claim 14, wherein the tips are formed from a material having high thermal conductivity.

17. Forceps as claimed in claim 14, wherein the metal tips are have a non-stick coating.

18. Forceps as claimed in claim 14, wherein the arms comprise a plastic outer body and the metal tips are embedded into the forward end of the plastic body.

19. Forceps as claimed in claim 18, wherein the body of the arms comprise an embedded conductor for supplying electrical current to the tips.

20. Forceps as claimed in claim 14 wherein the arms comprise a stiffener and a rearward section of each tip interconnects with a stiffener.

21. Surgical forceps comprising:
a pair of arms, each extending from a connecting member at a rearward end of the forceps to a tip at the forward end of the forceps; and
a pivot member disposed between the forward and rearward ends of the forceps and engaging the arms and wherein the axial position of the pivot member is adjustable.

22. Forceps as claimed in claim 21, wherein the forceps are electrosurgical forceps.
